SEQUENCE LISTING (4 sequences)

SEQ ID NO: 1 - DNA, 16 bp, Artificial
  Forward PCR Primer
gtaaaacgacggccag

SEQ ID NO: 2 - DNA, 17 bp, Artificial
  Reverse PCR primer
caggaaacagctatgac

SEQ ID NO: 3 - DNA, 26 bp, Artificial
  Forward LD PCR primer
ctcgggaagcgcgccattgtgttggt

SEQ ID NO: 4 - DNA, 28 bp, Artificial
  Reverse LD PCR primer
tatgctgagtgatatcccgcttaaccgg

(19) United States
(12) Patent Application Publication
(10) Pub. No.: US 2004/0175717 A1
(43) Pub. Date: Sep. 9, 2004

(54) Methods and kits for labeling and hybridizing cDNA for microarray analysis

(75) Inventor: Leonel Merwe van Zyl, Raleigh, NC (US)

Correspondence Address: JENKINS & WILSON, PA, 3100 TOWER BLVD, SUITE 1400, DURHAM, NC, 27707, US

(73) Assignee: North Carolina State University

(21) Appl. No.: 10/600,718

(22) Filed: Jun. 20, 2003

Related U.S. Application Data
(60) Provisional application No. 60/390,142, filed on Jun. 20, 2002.

(51) Int. Cl. C12Q 1/68; C12P 19/34

(52) U.S. Cl. 435/6; 435/91.2

(57) ABSTRACT

Methods and kits of the present invention provide an optimized system for cDNA microarray analysis. These systems include novel labeling, hybridization and stringency wash methods and compositions. These novel methods and kits provide robust and cost-effective protocols for labeling target cDNA for hybridization to probe nucleic acid immobilized onto a solid substrate, for highly efficient hybridization of target cDNA to probe nucleic acid, and for detection/visualization of these hybridizations wherein zero or almost zero background signal (i.e., “noise”) is generated. The high quality and sensitivity of the hybridization reactions thus provides statistically robust data for gene expression analysis. In one embodiment, methods for labeling target cDNA utilize a labeling mixture comprising 48 μM dATP, 48 μM dCTP, 48 μM dGTP, 6 μM dTTP and 6 μM of fluorescently labeled nucleotide selected from the group consisting of dUTP-Cy3™ and dUTP-Cy5™, and further utilize the Klenow fragment of DNA polymerase I to produce labeled target cDNA. In another embodiment, target cDNA that has been labeled according to the unique labeling mixture is hybridized to a microarray comprising a plurality of probe nucleic acid samples, wherein the microarray has been treated with a pre-hybridization buffer comprising 5×SSC, 1% BSA Fraction V and 0.1% SDS, where the SDS has a pH of between about 7.18 and about 7.25. In another embodiment, target cDNA and probe nucleic acid immobilized onto a microarray are hybridized in a buffer comprising polyA RNA, Calf Thymus DNA, 5×SSC, 5× Denhard's solution, 50% formamide, and 0.5% SDS, wherein the SDS has a pH of between about 7.18 and about 7.25. In another embodiment, a microarray comprising labeled target cDNA hybridized to probe nucleic acid is washed at least three times with post-hybridization buffers, wherein the first buffer comprises 1×SSC and 0.2% SDS, the second buffer comprises 0.1×SSC and 0.2% SDS, and the third comprising 0.1% SSC, wherein the SDS has a pH of between about 7.18 and about 7.25.

### RELATED APPLICATIONS

This application claims the benefit and priority of U.S. Provisional Patent Application No. 60/390,142, filed Jun. 20, 2002, which application is incorporated herewith in its entirety.

### GRANT STATEMENT

This invention was made in part with U.S. Government support under Grant Number DBI9975806 from the National Science Foundation. The U.S. Government has certain rights in the invention.

### FIELD OF THE INVENTION

The field of the invention is nucleic acid labeling and hybridization, particularly labeling and hybridization of nucleic acid targets for use in microarray-based hybridization assays.

### Table of Abbreviations

BSA—bovine serum albumin

cDNA—complementary DNA

CIA—chloroform/isoamyl alcohol

CTAB—cetyltrimethylammonium bromide

DMF—dimethylformamide

dATP—deoxyadenosine triphosphate

dCTP—deoxycytidine triphosphate

dGTP—deoxyguanosine triphosphate

DMSO—dimethylsulfoxide

DNA—deoxyribonucleic acid

dNTP—deoxynucleotide triphosphate

DTT—dithiothreitol

dTTP—deoxythymidine triphosphate

dUTP—2′-deoxyuridine 5′-Triphosphate

EDTA—ethylenediaminetetraacetic acid

EST—expressed sequence tag

h or hr—hour(s)

l or L—liter

LB—Luria-Bertani medium

M—molar

mg—milligram(s)

min—minute(s)

ml or mL—milliliter(s)

mm—millimolar

mRNA—messenger RNA

ng—nanogram

nM—nanomoles or nanomolar

PB—phosphate buffer

PBS—phosphate buffered saline

PCR—polymerase chain reaction

PEG—polyethylene glycol

RNA—ribonucleic acid

rpm—revolutions per minute

RT—room temperature

SD—standard deviation

SDS—sodium dodecyl sulfate

TBE—Tris-borate-EDTA buffer

U—enzymatic units

μg—microgram(s)

μl—microliter(s)

### BACKGROUND ART

Nucleic acid arrays have become an increasingly important tool in the biotechnology industry and related fields. Nucleic acid arrays, in which a plurality of nucleic acids are deposited onto a solid support surface in the form of an array or pattern, find use in a variety of applications, including drug screening, nucleic acid sequencing, mutation analysis, and the like.

One important use of nucleic acid arrays is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

Sequencing of genomes from various species has become part of daily life in molecular biology, and a large number of cDNA collections are publicly available. These high throughput DNA sequencing efforts made it possible to identify large numbers of genes. However, the nature and extent of molecular interactions of these genes and their respective products still remain the most fundamental question in biology. Information regarding genetic interactions is central to a better understanding of molecular structure and function, cellular metabolism, development of cells and tissues, response of organisms to their environments and as a fundamental basis for adaptation and evolution.

In the past several years, a new technology, called DNA microarrays, has attracted tremendous interest among scientists. DNA microarray has revolutionized the traditional way of “one gene per experiment” for the study of genetic interactions. The technique allows for massive parallel data acquisition and analysis. Parallelism greatly increases the speed and experimental progress and allows meaningful comparisons to be made between genes and gene products represented on the array.

Microarray experiments rely on the principle of hybridization (base pairing). These experiments are sometimes referred to as “reverse Northerns.” In Northern blots, RNA is blotted onto a filter hybridized with a probe to detect a particular species of mRNA as a distinct band or spot. In microarray hybridization, cDNAs are spotted onto nylon filters or glass slides and hybridized with a probe made from a mRNA population of interest. Usually, probes are made by reverse-transcribing mRNA into single-stranded cDNA in the presence of labeled nucleotides. The labeled probe, therefore, is a population of cDNA molecules representing the original mRNA population. Arrays are however, still limited with respect to quantitative measurements of mRNA abundance. Although inferences of significant differences in differential expression can be made with highly increased statistical power, it is still not possible to estimate actual mRNA abundance.

In many currently employed array based gene expression analysis protocols, differences in mRNA levels between two samples are detected and related to the expression level of different genes in the compared samples. Detection of different mRNA levels typically involves the steps of generating a target nucleic acid population that is representative of the mRNA population of the test sample. In other words, a population of target nucleic acids is generated where the population is indicative of the different mRNA levels that are originally present in the sample. The target nucleic acid population may be DNA or RNA and may have the sequence of the initial mRNA or the complement thereof. Following generation, the population of target nucleic acids is hybridized to an array of probe nucleic acids stably associated with the surface of a solid support. Since the sequence and location of each probe is known, any resultant hybridization complexes that form on the array surface between target and probe can be used to identify those genes that are expressed in the cell from which the initial mRNA sample was obtained. The intensity of the individual signals can also be used to at least semi-quantitatively determine the expression level of the detected genes. Since the methods require detection of target/probe complexes on the array surface, the target nucleic acids are generally labeled so that they can be detected.

In most cDNA array applications, high-density cDNA samples are deposited or “printed” onto small, discrete areas of solid substrates, sometimes referred to as slides or chips. These substrates can be glass slides of membrane filters and may optionally be coated. The cDNA samples, which are usually deposited by a robotic or automated system onto the substrate are immobilized onto the surface of the substrate to form a microarray. The microarray is then hybridized with a mixture of fluorescently labeled nucleic acids (probes) derived from a source of interest. After hybridization, the fluorescent markers are detected using a laser scanner. A pattern of gene expression is obtained by analyzing the signal emitted from each spot with digital imaging software that is generally commercially available. Differential analysis of the gene expression profile may be carried out in comparison to a control profile.

The terms “probes” and “targets” are used variably in the literature. As used herein, the term “probe” refers to nucleic acid (e.g., cDNA, oligonucleotides) immobilized onto the surface of the solid substrate while the term “target” refers to nucleic acid that is fluorescently labeled and then hybridized to the immobilized target DNA.

DNA microarray technology is useful in myriad applications, including the analysis of gene expression, monitoring changes in genomic DNA (e.g., in cancer development), mutation and polymorphism detection, gene discovery, genotyping, pathway analysis, and others that are still in the process of being delineated. There are technical obstacles that are common to all applications of cDNA technology.

Although the specific procedural steps carried out in the course of cDNA experiments may vary significantly, the scheme of cDNA analysis generally follows a course of seven broadly defined steps: (1) processing cDNA clones or oligonucleotides to generate nucleic acid suitable for printing onto substrate; (2) printing cDNA clones or oligonucleotides onto a substrate; (3) isolating sample RNA from which hybridizing targets are derived; (4) preparing the target cDNA from the mRNA (cDNA synthesis and labeling); (5) hybridization of target to the DNA arrayed on the substrate; (6) image acquisition of hybridization and (7) image analysis.

Clone sources generally include genomic or cDNA sequence data, or cDNA clones, or both. More recently, large scale sequencing of expressed sequence tags (EST) have increased the rate of gene discovery. For example, GENBANK presently has over one million human ESTs. Sequence-validated clone sets derived from EST sequence information are commercially available for several organisms (e.g., yeast, Arabidopsis thaliana, mouse, human, Caenorhabditis elegans, Drosophila melaogaster, numerous bacteria, and other organisms).

The clone sets useful in the creation of microarrays are so large that generally automation is required for sample dispensing and plate handling in the form of robotic printing is used to print arrays. These robotic systems may be used to directly transfer cDNA target samples from and to and between 96-well or 384-well sample plates, and may optionally be integrated with other automated systems. Numerous robotic printing systems are known and may be used in the practice of the present invention, including but not limited to the Robbins HYDRA WORK STATION™, the Beckman Coulter MULTIMEK™, the Qiagen BIOROBOT™, Tecan AG GENESIS™ sample processor, the Packard MultiPROBE™ and the Tomtec QUADRA™.

Numerous solid supports for microarrays, and methods for immobilizing cDNA targets onto the same, are known in the art. Chemically treated microscope glass slides are most widely used. Glass slides may be coated with amine or aldehyde surface chemistry, or DNA may be covalently bound to glass surfaces. Other solid surfaces include porous substrates such as nitrocellulose, nylon or acrylamide, alone or in conjunction with glass surfaces (e.g., nitrocellulose glass surfaces) are also used. Microarrays, with cDNA already arrayed onto a surface, are also available.

One the microarray has been printed or purchased, the next step is to hybridize a labeled target to the immobilized probe on the array. Typically, the targets for arrays are labeled representations of cellular mRNA pools isolated from various biological sources, such as cell cultures, tissues of model organisms, clinical biopsies, and histological samples. Target samples are generally prepared by isolating RNA, synthesizing cDNA and incorporating fluorescent dyes) and then hybridizing the probe molecule to the immobilized cDNA.

After hybridization, the DNA microarray is scanned to monitor the fluorescence of each probe that was successfully hybridized to the target. Most microarrays utilize two fluorophores, Cy3™ (green channel excitation) and Cy5™ (red channel excitation).

To generate a complete microarray image, it is necessary to acquire an image for each of the fluorophores. In general, two different scanning approaches are used: (1) sequential scanning, where one image is acquired at a time and then builds the ratio image after acquisition is completed, and (2) simultaneous scanning that acquires both images at the same time. Scanners are available from a variety of commercial sources, such as the GenePix 4000 (Axon Instruments), ScanArray 5000 (General Scanning, Inc.) GeneTac 1000 (Genomic Solutions), HP GeneArray Scanner (HewlettPackard), the Storm system (Molecular Dynamics) and the GMS 418 Array Scanner (Genetic Microsystems).

The objective of microarray analysis is to extract hybridization signals from each probe. Signals are measured either as absolute intensities for a given singular target, or as ratios of two probes with different fluorescent labels The ratio of two signals provides relative response ratios rather than an estimate of an absolute signal.

Once images are obtained in digitized form, they are subjected to further analysis using a variety of software programs. These programs attempt to provide a more accurate quantification of the intensity ratio. Background fluorescence, such as autofluorescence of the solid support, or non-specific binding of sample to the array, can be subtracted from the intensity of a feature. Subsequently, the mean, median and standard deviation of pixel intensities in each feature is determined and subjected to further detailed analysis. Image analysis programs are commercially available, and include GenePix Pro (Axon) and Scanalyze (available at http:/rana.stanford.edu/software/. Others include ArrayVision (Imaging Research Inc.), deArray (NHGR1), Imagene (BioDiscovery); TIGR Spoffinder (TIGR), MicroArraSuite (Scanlaytcs), GenExplore (Applied Maths), GeneData AG (Basel), Partek Pro 2000 (Partek Inc), and Spoffire.net Spoffire).

### SUMMARY OF THE INVENTION

One embodiment is a method of producing a population of labeled target cDNA, comprising combining a cDNA template with a mixture comprising 48 μM dATP, 48 μM dCTP, 48 μM dGTP, 6 μM dTTP and 6 μM of fluorescently labeled nucleotide selected from the group consisting of dUTP-Cy3™ and dUTP-Cy5™ to provide a nucleotide labeling mixture; adding a nucleic acid primer sufficient to prime the enzymatic generation of a population of target nucleic acids complementary to the cDNA template; and then reacting the primer and the nucleotide labeling mixture in the presence of the Klenow fragment of DNA polymerase I to produce labeled target cDNA.

Another embodiment is a method of hybridizing a population of target nucleic acids to an array made up of a plurality of probe nucleic acid samples stably associated with the surface of a solid support, said method comprising: generating said population of target nucleic acids by first combining a cDNA template with a mixture comprising 48 mM dATP, 48 mM dCTP, 48 mM dGTP, 6 mM dTTP and 6 mM of fluorescently labeled nucleotide selected from the group consisting of dUTP-Cy3™ and dUTP-Cy5™ to provide a nucleotide labeling mixture; then adding a nucleic acid primer sufficient to prime the enzymatic generation of a population of target nucleic acids complementary to the cDNA template; and reacting the primer and the nucleotide labeling mixture in the presence of the Klenow fragment of DNA polymerase I to produce labeled target cDNA; and then hybridizing said generated population of target nucleic acids to plurality of probe nucleic acid samples stably associated with the surface of a solid support.

Still a third embodiment is a kit for fluorescently labeling a nucleic acid, comprising a labeling mixture comprising dATP, dCTP, dGTP, dTTP and at least one of a fluorescently labeled nucleotide selected from the group consisting of dUTP-Cy3™ and dUTP-Cy5™, wherein the ratio of dATP to dCTP to dGTP to dTTP to dUTP-Cy3 or dUTP-Cy5 is 8:8:8:1:1. In one embodiment, the labeling mix is a 5× mixture, and ratio of concentrations of dATP to dCTP to dGTP to dTTP to dUTP-Cy3 or dUTP-Cy5 is 240 μM: 240 μM: 240 μM: 30 μM: 30 μM.

In one embodiment, the ratio of fluorescent labeled Cy3 and Cy5 dUTP/ to dTTP is unique, and advantageously provides a major cost savings and high reliability. In other embodiments, sodium dodecyl sulfate (SDS) used in prehybridization buffers, hybridization buffers, and post-hybridization washes has a pH ranging from 7.18 and about 7.25, and in still other embodiments, the sodium dodecyl sulfate (SDS) used in pre-hybridization buffers, hybridization buffers, and post-hybridization washes has a pH of 7.2

The present optimized microarray procedure advantageously produces superior quality spots (array images) with no background noise, due to the combination of several different optimized steps. The optimized nucleotide labeling mixture significantly reduces the concentration of fluorescently labeled d-UTP by more than half of that of other commercially available protocols, resulting in a much more efficient incorporation of labeled nucleotides.

In addition, we are using DNA Polymerase I, E. coli (Klenow fragment) for the indirect incorporation of fluorescently labeled d-UTP nucleotides. Klenow is superior with regards to the incorporation rate of nucleotides (dNTPs: deoxynucleoside triphosphates) as compared to all other commercially available reverse transcriptases (RT). For example, one unit of Klenow catalyzes the incorporation of 10 nmoles of dNTPs, compared to that of other commercially available reverse transcriptases, where 1 unit of enzyme only catalyzes the incorporation of 3 nmole of dNTPs in 30 minutes at 37° C. This results in a more qualitative representation of gene transcripts in a test sample, thus allowing the scientist to detect genes (transcripts) that are expressed at relatively low copy numbers.

The resulting high quality array images are also due to completely optimized hybridization, probe blocking and stringency wash procedures. This prevents false positive signals and removes all remaining chemical components that contribute to an increase in background noise. This is one of the key advantages of the optimized Klenow procedure compared to other microarray protocols currently available. Data is produced with no background noise (i.e., high signal-to-noise ratios).

Finally, the present inventor has made the surprising discovery that the pH of sodium dodecyl sulfate (SDS), a detergent compound commonly used in prehybridizing buffers, hybridizing buffers and post-hybridizing washes, is critical to reducing if not actually eliminating background noise. In the methods and kits described herein, solutions of SDS are brought to a pH ranging from about 7.18 to about 7.25, and optimally to a pH of 7.2, prior to the addition of SDS as a component in prehybridizing buffers, hybridizing buffers and post-hybridizing washes of the invention.

Thus, it is an object of the present invention to provide methods and kits for robust and cost effective protocols for labeling cDNA targets and for hybridization and for use in microarray analysis. The present invention provide significant improvements relative to any previously described procedure in terms of image quality, high signal/noise ration (approaching or reaching zero background noise), reliability, reproducibility and cost efficiency. These results have been confirmed by vigorous statistical analyses in various tissue types.

In repeated experiments, the optimization of the labeling, hybridization and stringency wash procedures resulted in essentially zero background noise. This is significant when considering that most statistical programs used for array analyses require complicated algorithms to subtract background noise in order to estimate true gene effects.

It is a further object of the invention to provide the artisan statistically robust data for gene expression analyses.

An object of the invention having been stated hereinabove, and which is addressed in whole or in part by the present invention, other objects will become evident as the description proceeds when taken in connection with the accompanying drawings as best described hereinbelow.

### BRIEF DESCRIPTION OF THE DRAWINGS

FIG. 1 provides two tables that compare the amount of fluorescent signal intensity and related fluorescent background noise of detected hybridization microarray spots that have been prepared by two different methods. In this table, “ch1” means intensity detected for “Channel 1,” or the Cy5™ or “red channel” of a fluorescent scanner. The data presented in the table on the left are the values generated for hybridization experiments performed with the Genisphere™ Microarray Kit. The data presented in the table on the right, labeled the “NCSU Method,” were generated using the presently described methods and reagents. Microarray spots produced using the presently described methods and reagents generated zero detectable background.

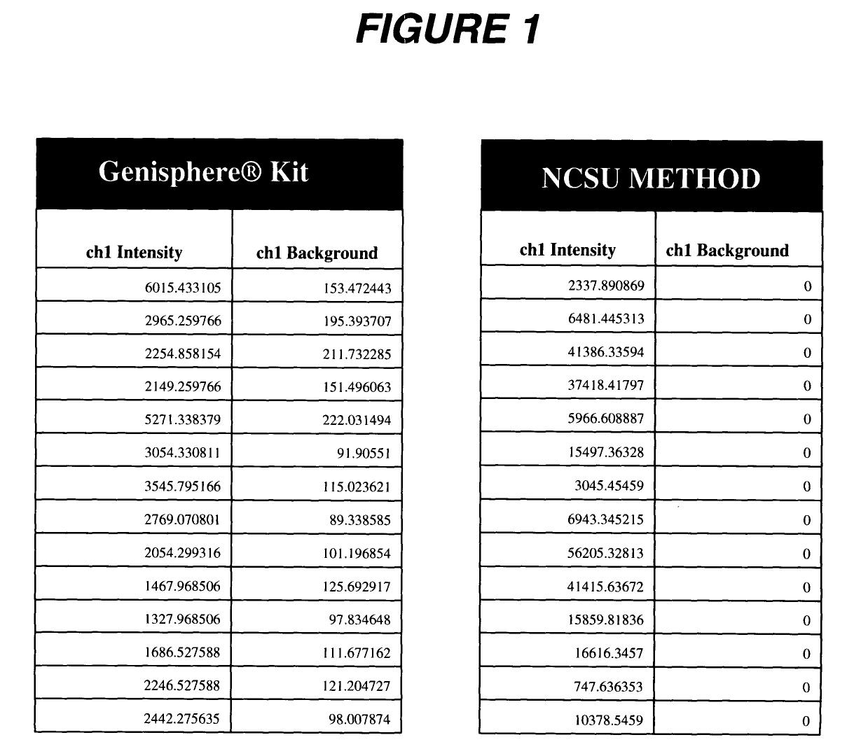

FIG. 2 is a scatter plot of log 2 intensities of microarray data generated using a commercially available microarray kit, with log 2 signal intensity value for the dye Cy3™ represented on the Y-axis and log 2 signal intensity value for the dye Cy5™ represented on the X-axis.

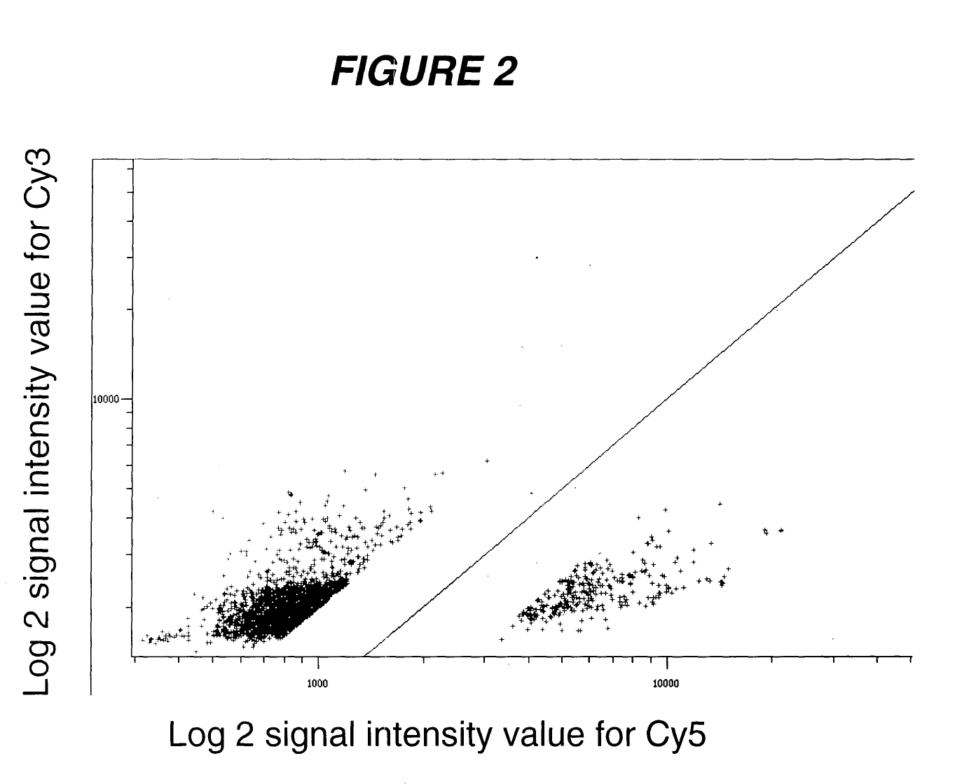

FIG. 3 is a scatter plot of log 2 intensities of microarray data generated using presently described methods and reagents, with log 2 signal intensity value for the dye Cy3™ represented on the Y-axis and log 2 signal intensity value for the dye Cy5™ represented on the X-axis.

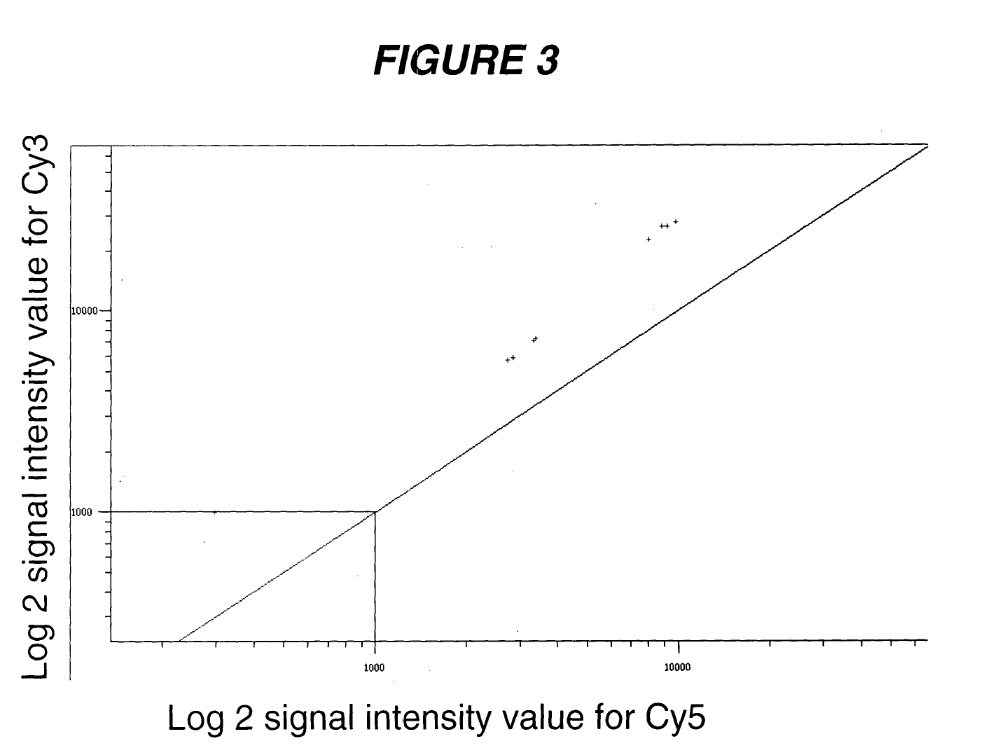

FIG. 4 is a scanned image of a cDNA microarray prepared with the Genisphere™ Microarray Kit according to the manufacturer's instruction, and visualized on the Cy5™ channel. The red color indicates that the presence of the fluorescent dye Cy5™ has been detected; the varying intensities of the color represents the amount of Cy5™ detected. The presence of diffuse and faint red color around the larger and more intense spots indicates the presence of undesirable background noise.

FIG. 5 is a scanned image of a cDNA microarray prepared with the presently described reagents and methods, and visualized on the Cy5™ channel. The probe cDNA samples immobilized on the solid substrate and the nucleic acid targets are the same as in the microarray shown in FIG. 4. The red color indicates that the presence of the fluorescent dye Cy5™ has been detected; the varying intensities of the color represents the amount of Cy5™ detected. The presence of undesirable background noise is dramatically reduced or non-existent compared with the background noise generated by the commercially available kit shown in FIG. 4. Moreover, the fluorescent signal indicating hybridization of target to probe is of greater intensity compared to the array shown in FIG. 4.

FIG. 6 is a scanned image of four 2304-element microarrays prepared with the presently described reagents and methods, and visualized on the Cy5™ channel (single-dye image). In FIG. 6, the color blue represents underexpression, green indicates intermediate expression, yellow indicates intermediate to high expression and red indicates very high expression.

FIG. 7 is a scanned image of the same four 2304-element microarrays shown in FIG. 6, but visualized on the Cy3™ channel (single-dye image). The colors that are visualized represent the same expression states as in FIG. 6.

FIG. 8 is an expanded image of an area of a microarray prepared and visualized with the presently described reagents and methods and visualized on the Cy5™ channel.

FIG. 9 is a graphical representation of the quality of raw intensity values generated from a microarray experiment is the most important factor influencing the interpretation of gene expression data. In FIG. 9, the green data points of the graph areas labeled “A” represent high expression fold change and highly significant results; the dark blue data points of the graph areas labeled “B” represent expression fold change smaller than 2, but highly significant; the light blue data points of the graph areas labeled “C” represent expression fold change larger than 2, but statistically insignificant; and the red data points of the graph areas labeled “D” represent expression fold change smaller than 2 and significantly insignificant.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All patents and publications mentioned herein are incorporated herein by reference for the purpose of describing and disclosing, for example, the cell lines, constructs, and methodologies that are described in the patents and publications, which might be used in connection with the presently described invention. The patents and publications discussed throughout the text are provided solely for their disclosure prior to the filing date of the present application. Nothing herein is to be construed as an admission that the inventors are not entitled to antedate such disclosure by virtue of prior invention.

Definitions

While the following terms are believed to be well understood by one of ordinary skill in the art, the following definitions are set forth to facilitate explanation of the invention.

As used herein and in the appended statements of the invention, the singular forms “a”, “and”, and “the” include plural referents unless the context clearly dictates otherwise. Thus, for example, reference to “a construct” includes a plurality of such constructs, and so forth.

The term “about”, as used herein when referring to a measurable value such as an amount of weight, time, dose, etc. is meant to encompass variations of in one embodiment ±20% or ±10%, in another embodiment ±5%, in another embodiment ±1%, and in still another embodiment ±0.1% from the specified amount, as such variations are appropriate to perform the disclosed methods.

The terms “nucleic acid material” and “nucleic acids” each refer to deoxyribonucleotides, ribonucleotides, or analogues thereof in either single- or double-stranded form. Unless specifically limited, the term encompasses nucleic acids containing known analogues of natural nucleotides that have similar properties as the reference natural or antisense nucleic acid. Thus “nucleic acids” includes but is not limited to DNA, cDNA, RNA, antisense RNA, and double-stranded RNA. A therapeutic nucleic acid can comprise a nucleotide sequence encoding a therapeutic gene product, including a polypeptide or an oligonucleotide.

Nucleic acids can further comprise a gene (e.g., a therapeutic gene), or a genetic construct (e.g., a gene therapy vector). The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “expression”, as used herein to describe a genetic construct, generally refers to the cellular processes by which a biologically active polypeptide or biologically active oligonucleotide is produced from a DNA sequence.

The term “construct”, as used herein to describe a genetic construct, refers to a composition comprising a vector used for gene therapy or other application. In one embodiment, the composition also includes nucleic acids comprising a nucleotide sequence encoding a therapeutic gene product, for example a therapeutic polypeptide or a therapeutic oligonucleotide. In one embodiment, the nucleotide sequence is operatively inserted with the vector, such that the nucleotide sequence encoding the therapeutic gene product is expressed. The term “construct” also encompasses a gene therapy vector in the absence of a nucleotide sequence encoding a therapeutic polypeptide or a therapeutic oligonucleotide, referred to herein as an “empty construct.” The term “construct” further encompasses any nucleic acid that is intended for in vivo studies, such as nucleic acids used for triplex and antisense pharmacokinetic studies.

The terms “bind”, “binding”, “binding activity” and “binding affinity” are believed to have well-understood meanings in the art. To facilitate explanation of the present invention, the terms “bind” and “binding” are meant to refer to protein-protein interactions that are recognized to play a role in many biological processes, such as the binding between an antibody and an antigen, and between complementary strands of nucleic acids (e.g. DNA-DNA, DNA-RNA, and RNA-RNA). Exemplary protein-protein interactions include, but are not limited to, covalent interactions between side chains, such as disulfide bridges between cysteine residues; hydrophobic interactions between side chains; and hydrogen bonding between side chains.

The terms “binding activity” and “binding affinity” are also meant to refer to the tendency of one protein or polypeptide to bind or not to bind to another protein or polypeptide. The energetics of protein-protein interactions are significant in “binding activity” and “binding affinity” because they define the necessary concentrations of interacting partners, the rates at which these partners are capable of associating, and the relative concentrations of bound and free proteins in a solution. The binding of a ligand to a target molecule can be considered specific if the binding affinity is about 1×10⁴ M^{'1} to about 1×10⁶ M⁻¹ or greater.

The phrase “specifically (or selectively) binds”, for example when referring to the binding capacity of an antibody, also refers to a binding reaction which is determinative of the presence of the antigen in a heterogeneous population of proteins and other biological materials. The phrase “specifically (or selectively) binds” also refers to selective targeting of a targeting molecule, such as the hybridization of a RNA molecule to a nucleic acid of interest under a set of hybridization conditions as disclosed herein below.

Preparation of Probe Nucleic Acid

Probe nucleic acid sequences of the invention can be derived from virtually any source. Typically, the probes will be nucleic acid molecules derived from representative locations along a chromosome of interest, a chromosomal region of interest, an entire genome of interest, a cDNA library, and the like. These probe oligonucleotides may be derived, for instance, from genomic clones, restriction digests of genomic clone, cDNA clones and the like. In some embodiments the probe nucleic acid sequences are derived from a previously mapped library of clones spanning a particular region of interest.

The choice of probe nucleic acids to use may be influenced by prior knowledge of the association of a particular chromosome or chromosomal region with certain disease conditions. Alternatively, whole genome screening to identify a new region subject to, e.g., frequent changes in copy number can be performed using the methods of the present invention. In these embodiments, target elements usually contain nucleic acid sequences representative of locations distributed over the entire genome. In some embodiments (e.g., using a large number of target elements of high complexity) all sequences in the genome can be present in the array.

Probe nucleotides used on the microarrays are typically prepared using previously genetically or physically mapped sequences.

Solid Supports and Surface Chemistry of Microarray Substrate

In one embodiment of the invention, the one or more nucleic acids from the subject of interest are immobilized on a solid support such that a position on the support identifies a particular nucleic acid. In the case of a set, constituent nucleic acids of the set can be combined prior to placement on the solid support or by serial placement of constituent nucleic acid at a same position on the solid support.

A microarray can be assembled using any suitable method known to one of skill in the art, and any one microarray configuration or method of construction is not considered to be a limitation of the present invention. Representative microarray formats that can be used in accordance with the methods of the present invention are described herein below.

The substrate for printing the array should be substantially rigid and amenable to immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman of Maidstone, United Kingdom) membrane.

Porous substrates (membranes and polymer matrices) permit immobilization of relatively large amount of probe molecules and provide a three-dimensional hydrophilic environment for biomolecular interactions to occur (Dubiley et al. (1997) Nuc Acids Res 25:2259-2265; Yershov et al. (1996) Proc Natl Acad Sci USA 93:4319-4918). A BIOCHIP ARRAYER™ dispenser (Packard Instrument Company of Meriden, Conn., United States of America) can effectively dispense nucleic acids onto membranes such that the spot size is consistent among spots whether one, two, or four droplets were dispensed per spot (Englert (2000) in Schena, ed., Microarray Biochip Technology, pp. 231-246, Eaton Publishing, Natick, Mass., United States of America).

A microarray substrate for use in accordance with the methods of the present invention can have either a two-dimensional (planar) or a three-dimensional (non-planar) configuration. An exemplary three-dimensional microarray is the FLOW-THRU™ chip (Gene Logic, Inc. of Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al. (1998) Science 282:2244-2246 and Steel et al. (2000) in Schena, ed., Microarray Biochip Technology, pp. 87-118, Eaton Publishing, Natick, Mass., United States of America.

Briefly, a FLOW-THRU™ chip (Gene Logic, Inc.) comprises a uniformly porous substrate having pores or microchannels connecting upper and lower faces of the chip. Probe nucleic acids are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767.

Surface Chemistry

The particular surface chemistry employed is inherent in the microarray substrate and substrate preparation. Immobilization of nucleic acids probes post-synthesis can be accomplished by various approaches, including adsorption, entrapment, and covalent attachment. Preferably, the binding technique does not disrupt hybridization activity.

For substantially permanent immobilization, covalent attachment is preferred. Since few organic functional groups react with an activated silica surface, an intermediate layer is advisable for substantially permanent probe immobilization. Functionalized organosilanes can be used as such an intermediate layer on glass and silicon substrates (Liu & Hlady (1996) Coll Sur B 8:25-37; Shriver-Lake (1998) in Cass & Ligler, eds., Immobilized Biomolecules in Analysis, pp.1-14, Oxford Press, Oxford, United Kingdom). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GMBS) that can bind maleimide to a primary amine of a probe. Procedures for using such linkers are known to one of skill in the art and are summarized by Hermanson (1990) Bioconiugate Techniques, Academic Press, San Diego, Calif. A representative protocol for covalent attachment of DNA to silicon wafers is described by O'Donnell et al. (1997) Anal Chem 69:2438-2443.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner (1997) in Vickerman, ed., Surface Analysis: The Principal Techniques, John Wiley & Sons, New York; Schena et al. (1995) Science 270:467-470). See also Worley et al. (2000) in Schena, ed., Microarray Biochip Technology, pp. 65-86, Eaton Publishing, Natick, Mass., United States of America.

For attachment of probe nucleic acids greater than about 300 base pairs, noncovalent binding is suitable. When using this method, aminosilanized slides are preferred in that this coating improves nucleic acid binding when compared to bare glass. This method works well for spotting applications that use about 100 ng/μl (Worley et al. (2000) in Schena, ed., Microarray Biochip Technology, pp. 65-86, Eaton Publishing, Natick, Mass., United States of America).

In the case of nitrocellulose or nylon membranes, the chemistry of nucleic acid binding chemistry to these membranes has been well characterized (Southern (1975) J Mol Biol 98:503-517); Maniatis et al. (1989) Molecular Cloning: A Laboratory Manual, Cold Spring Harbor Press, Cold Spring Harbor, N.Y.).

Arrays on solid surface substrates with much lower fluorescence than membranes, such as glass or quartz can achieve much better sensitivity. Substrates such as glass or fused silica are advantageous in that they provide a very low fluorescence substrate, and a highly efficient hybridization environment. Covalent attachment of the target nucleic acids to glass or synthetic fused silica can be accomplished according to a number of known techniques (described above). Nucleic acids can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). Quartz cover slips, which have at least 10-fold lower auto fluorescence than glass, can also be silanized.

Microarray Printing

A microarray can be constructed using any one of several methods available in the art, including but not limited to photolithographic and microfluidic methods. Ready-made, commercially available microarrays can also be employed.

As is standard in the art, a technique for making a microarray should create consistent and reproducible spots. Each spot is preferably uniform, and appropriately spaced away from other spots within the configuration. A solid support for use in the present invention preferably comprises about 10 or more spots, or more preferably about 100 or more spots, even more preferably about 1,000 or more spots, and still more preferably about 10,000 or more spots. Also preferably, the volume deposited per spot is about 10 picoliters to about 10 nanoliters, and more preferably about 50 picoliters to about 500 picoliters. The diameter of a spot is preferably about 50 μm to about 1000 μm, and more preferably about 100 μm to about 250 μm.

Representative techniques thus include: (1) Light-directed synthesis (Fodor et al. (1991) Science 251:767-773; Fodor et al. (1993) Nature 364:555-556; U.S. Pat. No. 5,445,934; and commercialized by Affymetrix of Santa Clara, Calif., United States of America); (2) Contact Printing (Maier et al. (1994) J Biotechnol 35:191-203; Rose (2000) in Shena, ed., Microarray Biochip Technology, pp. 19-38, Eaton Publishing, Natick, Mass., United States of America; Schena et al. (1995) Science 270:467-470; Mace et al. (2000) in Shena, ed., Microarray Biochip Technology, pp. 39-64, Eaton Publishing, Natick, Mass., United States of America); (3) Noncontact Ink-Jet Printing (U.S. Pat. No. 5,965,352; Theriault et al. (1999) in Schena, ed., DNA Microarrays: A Practical Approach, pp. 101-120, Oxford University Press Inc., New York, N.Y.); (4) Syringe-Solenoid Printing (U.S. Pat. Nos. 5,743,960 and 5,916,524); (5) Electronic Addressing (U.S. Pat. No. 6,225,059 and International Publication No. WO 01/23082); and (6) Nanoelectrode Synthesis (U.S. Pat. No. 6,123,819).

Probe elements of various sizes, ranging from 1 mm diameter down to 1 μm can be used. Smaller probe elements containing low amounts of concentrated, fixed DNA are used for high complexity comparative hybridizations since the total amount of sample available for binding to each probe element will be limited. Thus it is advantageous to have small array probe elements that contain a small amount of concentrated probe DNA so that the signal that is obtained is highly localized and bright.

In one particularly preferred embodiment, probe nucleic acid is spotted onto a surface (e.g., a glass or quartz surface). The nucleic acid is dissolved in a mixture of water and dimethylsulfoxide (DMSO), and spotted onto amino-silane coated glass slides.

Target Preparation

As with probe nucleic acid sequences, a wide variety of nucleic acids can be used as the source of the labeled nucleic acid sequences in the methods of the present invention. The labeled nucleic acid sequences may be prepared from, for example, genomic DNA representing the entire genome from a particular organism, tissue or cell type or may comprise a portion of the genome, such as a single chromosome.

To compare expression levels of a particular gene or genes, the labeled nucleic acid sequences can be derived from mRNA or cDNA prepared from an organism, tissue, or cell of interest. For instance, test cDNA or mRNA, along with mRNA or cDNA from normal reference cells, can be used to prepare labeled nucleic acid sequences which are hybridized to an array of oligonucleotides from a normalized cDNA library. In addition, labeled nucleic acid sequences made from genomic DNA from two cell populations can be hybridized to oligonucleotide microarray prepared from cDNA to detect those cDNAs that come from regions of variant DNA copy number in the genome.

The methods of the invention are suitable for comparing copy number of particular sequences in any combination of two or more populations of nucleic acid sequences. One of skill will recognize that the particular populations of sample nucleic acid sequences being compared is not critical to the invention. For instance, genomic or cDNA can be compared from two related species. Alternatively, levels of expression of particular genes in two or more tissue or cell types can be compared. As noted above, the methods are particularly useful in the diagnosis of disease.

Standard procedures can be used to isolate nucleic acids used as the source of the labeled nucleic acid sequences of the invention (either DNA or mRNA) from appropriate tissues (see, e.g., Sambrook, et al., Molecular Cloning—A Laboratory Manual, Third Edition, Cold Spring Harbor Laboratory, Cold Spring Harbor, N.Y. (2001)). Conventional methods for preparation of cDNA from mRNA can also be used.

The particular cells or tissue from which the source nucleic acids are isolated will depend upon the particular application. For example, for detection of abnormalities associated with cancer, genomic DNA is isolated from tumor cells. For prenatal detection of disease, fetal tissue is used.

Labeling of Target

The labels used in the invention may be incorporated into the nucleic acids by fluorescent nucleotides are incorporated into the amplified sequences using either the Klenow fragment of DNA Polymerase I.

Detectable labels suitable for use in the present invention include Cy3™ and Cy5™.

A fluorescent label is preferred because it provides a very strong signal with low background. It is also optically detectable at high resolution and sensitivity through a quick scanning procedure. In the present embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

The quality of sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner (1995) Nuc Acids Res 25:2923-2929). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al. (2000) in Shena, ed., Microarray Biochip Technology, pp. 65-86, Eaton Publishing, Natick, Mass., United States of America. Thus, it will be understood to one of skill in the art that labeling methods can be optimized for performance in microarray hybridization assay, and that optimal labeling can be unique to each label type.

Hybridization of Labeled Targets to Probes

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. In general, a signal to noise ratio of 2-fold (or higher) than that observed for a negative control probe in a same hybridization assay indicates detection of specific or substantial hybridization.

Standard hybridization techniques are used in the methods of the invention. Suitable methods are described in references describing CGH techniques (Kallioniemi et al., Science 258: 818-821 (1992) and WO 93/18186). Several guides to general techniques are available, e.g., Tijssen, Hybridization with Nucleic Acid Probes, Parts 1 and 11 (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985).

Generally, nucleic acid hybridizations comprise the following major steps: (1) immobilization of target nucleic acid sequences; (2) prehybridization treatment to increase accessibility of target DNA, and to reduce nonspecific binding; (3) hybridization of the mixture of nucleic acid sequences to the nucleic acid on the solid surface; (4) post-hybridization washes to remove nucleic acid fragments not bound in the hybridization and (5) detection of the hybridized nucleic acid fragments. The reagent used in each of these steps and their conditions for use vary depending on the particular application.

Hybridization Detections and Image Acquisition

Standard methods for detection and analysis of signals generated by the labeled nucleic acids can be used. The particular methods will depend upon the labels used in the labeled nucleic acids. Generally, fluorescent labels are preferred. Thus, methods suitable in fluorescence in situ hybridization (FISH) are suitable in the present invention. For instance, the nucleic acid arrays can be imaged in a fluorescence microscope with a polychromatic beam-splitter to avoid color-dependent image shifts. The different color images are acquired with a CCD camera and the digitized images are stored in a computer. A computer program is then used to analyze the signals produced by the array. Methods of visualizing signals are described, for instance, in Kallioniemi et al., supra and in WO 93/18186.

To facilitate the display of results and to improve the sensitivity of detecting small differences in fluorescence intensity, a digital image analysis system is preferably used.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al. (1996) The International Society of Optical Engineering 2705/63.

Analysis of Detectable Signals From Hybridizations

Numerous software packages have been developed for microarray data analysis, and an appropriate program can be selected according to the array format and detection method. Some products, including ARRAYGAUGE™ software (Fujifilm Medical Systems Inc. of Stamford, Conn., United States of America) and IMAGEMASTER ARRAY 2™ software (Amersham Pharmacia Biotech of Piscataway, N.J., United States of America), accept images from most microarray scanners and offer substantial flexibility for analyzing data generated by different instruments and array types. Other microarray analysis software products are designed specifically for use with particular array scanners or for particular array formats. A survey of currently available microarray analysis software packages can be found in Brush (2001) The Scientist 15(9):25-28. In addition, the guidance presented herein provides for the development of software and/or databases by one of ordinary skill in the art, to facilitate analysis of data obtained by performing the method of the present invention.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Preparation of dNTPs

An optimized, 5× Labeling Nucleotide Master Mix with 1:1 ratio of dTTP to Cy3/5dUTP by mixing the following reagents in a 1.5 ml Microfuge tube:

| | | Final |
|---|---|---|
| Nucleotide | Volume | Concentration |
| dATP (1200 μM) | 50 μl | 240 μM |
| dCTP (1200 μM) | 50 μl | 240 μM |
| dGTP (1200 μM) | 50 μl | 240 μM |
| dTTP (150 μM) | 50 μl | 30 μM |
| d-UTP Cy3/5 (150 μM) | 50 μl | 30 μM |
| Total | 250 μl | |

The 5× Labeling Nucleotide Master Mix is made according to the following protocol:

1) Preparation of 1200 μM of each dNTP

a) Take 5 μl of each of the 100 mM dATP, dGTP, dCTP, and dTTP

b) To each dNTP add 412 μl H₂O

i) 5 μl of 100 mM dATP+412 μl H₂O

ii) 5 μl of 100 mM dGTP+412 μl H₂O

iii) 5 μl of 100 mM dCTP+412 μl H₂O

iv) 5 μl of 100 mM dGTP+412 μl H₂O

2) Preparation of 150 μM dTTP

a) Take 7 μl of the 1200 μM dTTP solution and add it to 49 μl H₂O

i) 7 μl of the 1200 μM dTTP+49 μl H2O

3) Calculation of the concentration of Cy dye

a) Each of the Cy Dyes (Cy3 and Cy5) normally 25 nM and only 25 μl

i) Therefore 1 nM/μl

ii) 1 nM=20 μM

4) Preparation of a 150 μM solution

a) Take 7.5 μl of Cy3 or Cy5 stock and add to 42.5 μl H₂O

i) 7.5 μl Cy3+42.5 μl H2O=150 μM

ii) 7.5 μl Cy5+42.5 μl H2O=150 μM

5) Preparation of final mix (5× mix)

a) Add 50 μl of each of the 1200 μM dATP, dCTP, and dGTP

b) Add 50 μl of the 150 μM dTTP

c) Add 50 μl of the 150 μM Cy3 or Cy5

d) The total volume should be 250 μl

e) Use 20 μl of this for 50 ng DNA

### Example 2

### PCR Amplification and Probe Clone Preparation Protocol for Microarrays

cDNA clone inserts were amplified by PCR from plasmid DNA or directly from clones in culture. For the analysis of expression in most eukaryotes, expressed sequence tag (EST) data are used. EST's are single pass, partial sequences of cDNA clones and are used extensively for gene discovery and mapping in humans and other organisms. In general, EST minipreps were kept at 4° C. and were used in PCR reactions instead of amplifying from bacterial cultures.

If no plasmid stocks are available, plasmids may be isolated using the following protocol:

1.1. Selected clones are inoculated into 96-well blocks (Qiagen) containing 1.3 ml Magnificent Broth (MB) and 100 μg/ml Ampicillin.

1.2. Incubate for 16 hr at 37° C. in a shaker incubator (220 rpm)

1.3. Isolate plasmids using R.E.A.L. Prep 96 Plasmid Kit (QIAGEN) or Qiaprep 96 Turbo Miniprep Kit (Qiagen Cat # 27191)

Alternatively, direct colony PCR may be used to isolate plasmids, according to the following protocol:

2.1. Selected clones are incubated into 96-well blocks (Qiagen) containing 1.2 ml LB/Ampicillin (50 μg/ml)

2.2. Incubate for 16 hr at 37° C. in a shaker incubator (200 rpm).

2.3. Following overnight growth, 5 μl culture suspension are transferred into 96-well Falcon U-bottom plates (BD Biosciences) containing 95 μl Milli-Q-treated water (Millipore)

2.4. Microtiter plates are then incubated at 95° C. for 10 min in order to lyse the cells and release the plasmid clones

2.5. Before PCR, cellular debris is removed by centrifugation at 1200×g for 3 min

2.6. Use 2 μl culture supernatant for each PCR reaction

Prepare PCR's from NSF plasmid stock plates according to the following protocol:

3.1. Step 1: If 96-well plates (MJ Research) containing plasmid stocks are dried out, re-dissolve in 10 μl molecular grade water (Sigma, Cat #: W4502), leave at 4° C. overnight, vortex for a few seconds, quick spin to collect solution and store at −20° C.

3.2. Step 2: Plasmid dilutions: Dilute 1 μl plasmid stock in 99 μl Sigma H₂O. Vortex briefly and store at −20° C. Quick-spin to collect solution at bottom. If you are going to work with a certain plate multiple times, rather aliquot into smaller volumes. Important: Quick-spin every time after thawing diluted plasmid stocks in order to prevent cross contamination.

### Example 3

### PCR Amplification

Clone inserts are amplified in 50 μl reactions in 96-well reaction plates (MJ Research). A reaction master mixture is usually prepared for each reaction plate: Master mixtures are usually prepared with a 5% excess to incorporate pipetting error. The chemicals needed for PCR as follows:

4.1.1. Taq DNA polymerase, 5 units/μl: Roche Molecular Biochemicals; Cat # 1596594 (10×250 units). A 10× conc. PCR buffer containing 15 mM MgCl2 is supplied with enzyme.

4.1.2. PCR nucleotides: Set of Deoxy-Nucleotides: Roche Molecular Biochemicals: Cat # 1969064. Important: Prepare a 1 ml 10 mM stock by adding 100 μl of each of the dNTP's (dATP, dGTP, dCTP, DTTP) to 600 μl of water.

4.1.3. Primers:

| | |
|---|---|
| M13 Forward Amine: | 5′-GTA AAA CGA CGG CCA G-3′ |
| M13 Reverse Amine: | 5′-CAG GAA ACA GCT ATG AC-3′ |
| 5′-LD primer: | 5′-CTC GGG AAG CGC GCC ATT GTG TTG GT-3′ |
| 3′-LD primer: | 5′-TATGCT GAG TGA TAT CCC GCT TAACCGG-3′ |

A 100 μM stock solution is prepared by adding the exact amount of water provided to make a final concentration of 100 μmol/μl (usually provided on tubes). We usually prepare 1 ml of a 10 μM stock solution: ex: CV=CV: 100 μM×V=10 μM×1000 μl (required volume). Thus, the total volume needed from a 100 μM stock to prepare 1 ml (10 μM stock) is 100 μl primer+900 μl H₂O Water: Molecular grade water from Sigma (Cat # W4502)

4.2. Reaction Mixtures

4.2.1. Reaction mixture for a 50 μl reaction using diluted plasmid stocks (1 μl plasmid/99 μl water)

| | | |
|---|---|---|
| ddH2O | 39.1 | μl |
| 10 × PCR buffer | 5 | μl |
| dNTP (from 10 mM stock) | 1 | μl |
| Forward Primer (from 10 μM stock) | 1 | μl |
| Reverse Primer (from 10 μM stock) | 1 | μl |
| Taq (5 U/μl) | 0.4 | μl |
| Plasmid sample | 2.5 | μl |
| Total | 50 | μl |

4.2.2. Reaction mixture for direct colony PCR

| ddH2O | 41.6 | μl |
|---|---|---|
| 10 × PCR buffer | 5 | μl |
| dNTP (from 10 mM stock) | 1 | μl |
| Forward Primer (from 10 μM stock) | 1 | μl |
| Reverse Primer (from 10 μM stock) | 1 | μl |
| Taq (5 U/μl) | 0.4 | μl |
| Bacteria below | see description | |
| Total | 50 | μl |

A micropipette tip is used to touch the bacterial colony that has been grown overnight on LB/ampicillin agar plates. To ensure proper mixing, submerge the tip into the prepared PCR mixture and pipette up and down for about 3-5 times and start PCR cycling.

4.2.3. Master mixture for 100 PCR reactions (1×96-well plate), using diluted plasmid stock solutions:

| | | |
|---|---|---|
| ddH2O | 3910 | μl |
| 10 × PCR buffer | 500 | μl |
| dNTP (from 10 mM stock) | 100 | μl |
| Forward Primer (from 10 μM stock) | 100 | μl |
| Reverse Primer (from 10 μM stock) | 100 | μl |
| Taq (5 U/μl) | 40 | μl |
| Total | 5000 | μl |

4.2.4. Master mixture for 100 PCR reactions (1×96-well plate), using direct colony PCR

| | | |
|---|---|---|
| ddH2O | 4160 | μl |
| 10 × PCR buffer | 500 | μl |
| dNTP (from 10 mM stock) | 100 | μl |
| Forward Primer (from 10 μM stock) | 100 | μl |
| Reverse Primer (from 10 μM stock) | 100 | μl |
| Taq (5 U/μl) | 40 | μl |
| Total | 5000 | μl |

5. PCR cycling protocol:

Reactions are amplified in a thermocycler from MJ Research, Waltham, Mass., USA.

5.1. PCR conditions for M13 Forward and Reverse primers

Program name: AMP-INS

| Conditions | | | |
|---|---|---|---|
| Step 1 | 94° C. | 1 | sec |
| Step 2 | 94° C. | 30 | sec |
| Step 3 | 57° C. | 1 | min |
| Step 4 | 72° C. | 4 | min |
| Step 5 | go to Step 2 | | |
| | for 34 cycles | | |
| Step 6 | 72° C. | 10 | min |

| | | |
|---|---|---|
| Step 7 | 4° C. | forever |

5.2. PCR conditions for LD—Forward and Reverse primers

Program name: LD-INS

| Conditions | | | |
|---|---|---|---|
| Step 1 | 94° C. | 1 | min |
| Step 2 | 94° C. | 30 | sec |
| Step 3 | 68° C. | 3 | min |
| Step 4 | go to Step 2 | | |
| | for 34 cycles | | |
| Step 5 | 68° C. | 3 | min |

| | | |
|---|---|---|
| Step 7 | 4° C. | forever |

All plates are covered with rubber mats (MJ Research or Perkin & Elmer) and start reaction. After completion, do a quick spin down of all the plates in order to collect condensation and store at 4° C. (2) Keep an archive off all PCR products by diluting 1 μl PCR product in 99 μl TE buffer. Store at −20° C. Use 2 μl of this solution for follow-up PCR reactions.

### Example 4

### Protocol for Gel Electrophoresis

This step is necessary to confirm both quantity and quality of the PCR reactions. The primary criterion for quality in this case is the presence of a single band. PCR reactions that contain two equally abundant products are considered more likely to have arisen from contaminated templates.

6.1. When using the large gel trays:

6.1.1. Prepare 5 liters of 0.5×TBE (250 ml 10×TBE+4750 ml H₂O). Most of this are used as running buffer

6.1.2. To pour an 1% agarose gel: Combine 4 g agarose+400 ml 0.5 TBE

Melt in microwave and after cooling, add 12 μl ethidium bromide to gel solution

pour gel

6.1.3. To run gels: Take 3 μl PCR product, add 1.5 μl of 6× gel loading solution+5 μl water—mix by pipetting up and down.

6.1.4. In order to prepare a molecular weight marker solution, add 3 μl (3 pg) of 1 kb ladder, 6 μl of 6× loading dye and 27 μl water. For gel running, load 6 μl of this solution (0.5 μg of 1 kb ladder). The 1.6 kb band in Gibco's 1 kb ladder contains 10% of the total mass of DNA in your sample. Thus, 0.5 μg of the 1 kb ladder contains 50 ng of the 1.6 kb band. The amount of DNA in 3 μl of your sample should be roughly 3× this amount, or at least the same.

6.1.5. Run gel at 180V for 2 hrs.

6.1.6. Document results by printouts and save gel files for future records.

6.2. Chemicals needed:

6.2.1. 0.5 M EDTA: Add 186.1 g of disodium ethylenediaminetetra-acetate.2H₂O to 800 ml H₂O. Stir vigorously on a magnetic stirrer. Adjust pH to 8.0 with NaOH (+−20 g of NaOH pellets). Sterilize by autoclaving. A 50 ml stock should be more than enough, use 9.3 g of disodium ethylenediaminetetra-acetate.2H2O+40 ml H₂O+1 g NaOH: should be pH 8

6.2.2. 10×TBE (Tris-borate): 108 g Tris base+55 g boric acid+40 ml of 0.5 M EDTA (pH 8), dissolve in 800 ml H₂O and make up to 1 liter.

6.2.3. Buy 10×TBE (GibcoBRL Cat# 15581-028) Dilute

7. DNA PCR cleanup.

The Millipore filtration system (Cat# MANUO3010) is used.

7.1. Procedure:

7.1.1. Place 96 well plate in filtration manifold system, and remove the plastic lid.

7.1.2. Add 100 μl PCR mixture to the individual wells

7.1.3. Apply vacuum to filter, following the TIGR approach. They apply 15″ Hg pressure for 10 minutes.

7.1.4. Add 30 μl water (MilliQ or Sigma water in our case

7.1.5. Vacuum dry for 10 minutes

7.1.6. Add an additional 30 μl Sigma water

7.1.7. Vacuum dry for another 10 minutes.

7.1.8. Remove plate from manifold and

7.1.9. Add (redissolve) DNA in 40 μl Sigma water.

7.1.10. You can either let the DNA re-dissolve overnight at 4° or you can incubate for 2 hr at 4° C., place foil lid on PCR plate.

7.1.11. Manually transfer the 40 μl water containing DNA to a clean 96 well PCR plate (pipetting up and down several times).

8. Printing solution:

8.1. add to the 40 μl DNA, equal volumes of DMSO, 40 μl. (Using Hydra 96 Program: 40 μl transfer reservoir to AB0900). Or, in case of starting with 200 μl PCR product use program: 80 μl transfer reservoir to ABO900

8.2. Transfer 40 μl to 384, using program: Transfer 40 μl AB0900 to Genetix

9. Printing and post printing treatment:

9.1. Corning CMT-GAPS slides are used (Cat# 40-003). Printer: Affymetrix 417 arrayer and follow printer procedure.

9.2. After print is completed crosslink DNA to slides using the UV-cross-linker (250 mJ).

9.3. Also important is to mark one slide with a diamond marker to indicate where DNA was printed

9.4. After crosslinkage, bake slides at 75° C. for two (2) hours

9.5. Place in slide racks and store in the dark until use.

10. RNA Extractions Protocol

10.1. RNA Extractions (for a 5 g sample)

The following supplies and chemicals are used:

a. Liquid Nitrogen

b. Dry Ice

C. Extraction Buffer

d. SSTE Buffer

e. 10M LiCl

f. CIA

g. DEPC treated dH₂O

h. Coffee grinder and mortar and pestle

i. 65 degree Celsius water bath

j. Mercaptoethanol

k. Ice

10.2. Day 1:

10.2.1. Pipette 25 ml of extraction buffer (5 ml/g of tissue) to a falcon and place the tube into a 65 degree water bath to get up to temp. Add 500 μl Mercaptoethanol to the extraction buffer (2% v/v). (see recipe book)

10.2.2. Use dry ice to get coffee grinder cold enough so that your sample will not thaw while grinding it. Once sample is ground into a fine powder transfer the sample quickly to the mortar and pestle and add liquid nitrogen. Start to grind sample with pestle to get it even finer.

10.2.3. Then quickly transfer to the 65° C. extraction buffer/mercaptoethanol and shake vigorously. Vortex and place back at 65° C. for 5 min.

10.2.4. Add an equal amount of CIA and shake well

10.2.5. Centrifuge tubes at 10,000 rpm for 20 minutes at 4 degrees Celsius after each CIA extraction.

10.2.6. Pipette off clear supernatant into a new falcon tube. Be sure to get as much of the supernatant as possible with out contaminating with lower levels of aqueous material. Also be sure to measure the amount that you pipette of to continue onto the next step. Work at room temperature.

10.2.7. Repeat steps 10.2.4 to 10.2.6 an additional 2 times. Work at room temperature.

10.2.8. Calculate ¼ volume of the supernatant and add that amount in 10M LiCl. Mix gently by inverting several times.

10.2.9. Place in 4 oC-degree freezer overnight (or at least 6 hours). The longer it sits in 4oC the better yields you will get.

10.3. Day 2:

10.3.1. Remove tubes from the 4oC degree freezer and centrifuge at 10,000 rpm for 30 minutes at 4 degrees Celsius.

10.3.2. You should have a clear pellet after centrifuging. As a precaution pull off supernatant and transfer to another tube.

10.3.3. Resuspend pellet in 500 μl SSTE. Be sure to completely redisolve pellet. Do this by pipetting up and down the sides of the tube. Place on ice. NOTE: that if you are selecting polyA+ RNA dissolve the pellet in 0.5% SDS instead of SSTE and proceed directly to selection. Do NOT work on ice.

10.3.4. Transfer the 500 μl of SSTE and dissolve pellet into a 1.5 ml ependorf tube.

10.3.5. Add an equal amount of CIA (500 μl) to tube and mix by pipetting up and down. *Do not place back on ice.

10.3.6. Centrifuge for 10 minutes at max speed at 4 degrees Celsius.

10.3.7. Carefully pull off (and keep) as much of the aqueous layer as possible and transfer to a new eppendorf tube.

10.3.8. Add a double volume (1 ml) of Rnase free ethanol and mix by inverting the tube.

10.3.9. Place in the −70-degree freezer for 1-2 hours or overnight.

10.3.10. Centrifuge for 30 minutes at max speed at 4 degrees.

10.3.11. Dump supernatant and look for pellet.

10.3.12. Wash pellet by adding 200 μl of 70% ethanol and spin briefly.

10.3.13. Dump supernatant and dry tubes in speed vac (usually takes about 5 minutes).

10.3.14. Once dry, add 50-200 μl of Rnase free water and allow pellet to redissolve by setting at room temperature.

10.3.15. Once pellet is redissolved, determine quality of RNA by running a 1% agarose gel: use 2 μl of sample+2 μl of loading dye+2 μl of Rnase free water, mix and load into gel.

10.3.16. Also measure concentration of RNA using SpectraMax. In one well add 300 μl of RNase free water and to another add 3 μl of sample and 297 μl of Rnase free water.

Read at 260 and 280. Calculate the concentration as follows:

{A260}×(40 OD)×(100 dilution)/1000=ug/μl of RNA.

### Example 5

### Microarray Procedure

All incubations steps were carried out in a MJ Research MiniCycler. Important: Use 20 ug of total RNA as start up target concentration.

1. 1^{st} Strand cDNA Synthesis

| | Sample #1 | Sample #2 | Sample #3 |
|---|---|---|---|
| RNA (20 ug) | X | X | X |
| Oligo dT (500 ng/ul) | 2 ul | 2 ul | 2 ul |
| 10 mM dNTP | 2 ul | 2 ul | 2 ul |
| Sigma Water | Y | Y | Y |
| Total | 24 ul | 24 ul | 24 ul |

Note:
‘X’ is the amount of sample, which is dependent on the sample's concentration, and should not exceed 20 ul.
‘Y’ is the amount of water that needs to be added to bring the volume up to 24 ul.

For all incubation steps use PCR machine (miniCycler) by MJ Research.

1.1. Add the above listed ingredients to the RNA sample in the following order: Oligo dT, 10 mM dNTP, and then the Sigma water. Mix well by pipetting.

1.2. Incubate at 65 degree's Celsius for 5 minutes.

1.3. Place immediately onto ice and chill for 1 minute.

1.4. Centrifuge tubes briefly to gather all ingredients to the bottom of the tube.

1.5. Add the following ingredients:

a. 8 μl of 1 st Strand Buffer

b. 4 μl of 0.1M DTT

c. 2 μl of Rnase Out (40 U/μl)

d. Mix gently by pipetting up and down.

1.6. Centrifuge tubes briefly and place on ice.

1.7. Incubate at 42 degrees Celsius for 2 minutes, do not place back on ice.

1.8. Add 2 μl of Superscript II (200 U/μl) and mix gently with pipette then quick spin.

1.9. Incubate at 42 degrees Celsius for 50 minutes.

1.10. Incubate at 70 degrees Celsius for 15 minutes

1.11. Centrifuge briefly and then place on ice.

1.12. Total volume should equal 40 μl.

2. Precipitation

2.1. Add 1 volume (40 μl) of 2-propanol and mix well by pipetting up and down. Then quick spin.

2.2. Incubate at −70 degrees Celsius for 3 hours. Note that samples can sit overnight at −20 degrees Celsius. Overnight is preferred.

2.3. Centrifuge samples for 30 minutes at 14,000 rpm at 4 degrees Celsius.

2.4. Discard supernatant using a pipette.

2.5. Add 200 μl of 70% ethanol, and tilt to mix.

2.6. Centrifuge for 10 minutes at 14,000 rpm at 4 degrees Celsius.

2.7. Discard supernatant and dry tubes in speed vac (−5 to 10 minutes).

2.8. Once dried, add 53 μl of Sigma water and mix slowly by pipetting and place on ice. (Then proceed to run a 1% agarose gel. Do so by using 1 μl of sample added to 2 μl of loading dye and 4 μl of Sigma water.)

3. Labeling

3.1. Add 15 μl of Spiking control mix (Sp 1, 2, 3 and 4) (with concentration of 0.25 ng/μl each). Mix by pipetting.

3.2. Centrifuge briefly and place back on ice.

3.3. Denature the DNA at 95 degrees Celsius for 5 minutes.

3.4. Place on ice immediately and allow to cool.

3.5. Spin briefly and place back on ice.

The rest of this procedure is to be done in minimal light due to light sensitivity of Cy3 & Cy5.

3.6. Add 20 μl of the 5× Master Nucleotide Labeling described in Example 1 mix by pipetting and place on ice.

3.7. Add 10 ul of 10× Hexanucleotide primer (Roche cat# 1277-081), mix well by pipetting up and down very slowly, place back on ice.

3.8. Add 2 μl of Kleenow (5U/μl: Amersham Biosciences Cat# 2141-Y)), mix well by pipetting up and down very slowly. Place back on ice and cover with foil. You should have a final volume of 100 μl.

3.9. Incubate in the dark at 37 degrees Celsius for 1 hour.

4. Probe Cleanup (Do all steps in minimal light)

4.1. Prepare tubes by combining purple column with eppendorf tube, given in the kit.

4.2. Combine both cDNA's of Cy3 and Cy5. (200 μl) Probe cleanup is done by using the QIAquick PCR Purification Kit (cat# 28104), available from QIAGEN.

4.3. Add 1000 μl of Buffer PB and mix well by pipette. Total volume ˜1200 μl

4.4. Add 800 μl of above solution to the purple column.

4.5. Spin at 13000 rpm for 1 minute.

4.6. Discard flow through.

4.7. Add remaining solution from step 3 to purple column.

4.8. Spin at 13000 rpm for 1 minute.

4.9. Discard flow through.

4.10. Add 750 μl of Buffer PE to the purple column.

4.11. Spin at 13000 rpm for 1 minute.

4.12. Discard flow threw.

4.13. Add another 750 μl of Buffer PE to the purple column.

4.14. Spin at 13000 rpm for 1 minute.

4.15. Discard flow threw.

4.16. Spin an additional minute at 13000 rpm.

4.17. Label new 1.5 ml eppendorf tubes

4.18. Transfer purple column to new 1.5 ml eppendorf tube.

4.19. Add 30 μl of Buffer EB to the purple column.

4.20. Incubate for 1 minute.

4.21. Spin at 13000 rpm for 1 minute.

4.22. Add another 30 μl of Buffer EB to the purple column.

4.23. Incubate for 1 minute.

4.24. Spin at 13000 rpm for 1 minute.

4.25. Dry resuspended labeled DNA in speed vac for 60 minutes or until dry.

5. Preparation for pre-hybridization of slides

5.1. Start pre-hybridization halfway thru the drying process of the labeled probe.

5.2. Prepare 400 ml of fresh pre-hybridization buffer:

a. 100 m of 20×SSC (final concentration is 5×SSC)

b. 4 ml of 10% SDS, pH 7.2 (final concentration is 0.1% SDS)

c. 4 g of BSA, Fraction V (final concentration is 1%)

d. 296 ml of Sigma water

Note: To dissolve BSA, place solution into a 42 degree Celsius water bath.

5.3. Once dissolved, transfer 50 ml of the pre-heated (42° C.) pre-hybridization buffer into a 50 ml falcon tube.

5.4. Place printed slide to be hybridized into each tube.

5.5. Gently tilt for 2 minutes to remove any unbound DNA.

5.6. Pre-hybridize slides for exactly 45 minutes at 42 degrees Celsius.

5.7. During pre-hybridization prepare Falcon tubes for washing slides. Use 3-50 ml Falcon tubes for every 4 slides. 2 containing 50 ml sigma water and 1 containing 50 ml 2-Propanol.

5.8. Remove slide from pre-hybridization buffer and dip 5 times into the first falcon tube containing Sigma water. Then repeat with second tube containing Sigma water. (Sigma Cat# W4502). Touch corner of slide to side of tube to remove excess water. Important: slide should never be allowed to dry between the washes.

5.9. Remove from water and dip 5 times in 2-propanol to remove excess water.

5.10. Place in slide rack and dry by centrifuging at 500 rpm for 5 minutes.

5.11. Prepare incubation chambers for slides by pipetting 10 μl of Sigma water into provided slots on both sides.

5.12. Add slides to chambers and record bar codes according to experimental design.

5.13. Immediately continue with hybridization procedure. Pre-hybridized slides are used within one hour.

6. Hybridization of slides

6.1. Prepare 400 ul of fresh hybridization buffer following the following recipe:

a. 20 ul 10% SDS, pH 7.2 (0.5% final conc. Sigma Cat# L4522)

b. 100 ul 20×SSC (5×SSC final conc. Sigma Cat# S6639)

c. 40 ul 50× Denhards (5× Denhards final conc. Sigma Cat# D2532))

d. 200 ul Formamide (50% Formamide final conc.Sigma Cat# F9037)

e. 40 ul Sigma water

f. 20 ul of a 10 ug/ul Poly A RNA (Amersham Biosciences 27-4110-01)

g. 22 ul of 9.1 ug/ul Calf Thymus DNA. (Sigma Cat# D8661)

6.2. Add 40 ul of the above solution to each of your dried, labeled probes. Redisolve by slowly pipetting up and down and quick spin

6.3. Denature probes for 2 minutes at 95 degrees Celsius.

6.4. Centrifuge briefly.

6.5. Add hybridization solution in the middle of the printed area of the slide

6.6. Place a cover slip (Schleicher & Schuell Cat# 10484905) on top gently. Inspect for bubbles and gently press on coverslip with pipette tip to remove major bubbles.

6.7. Seal into Corning incubation chamber and hybridize in a 42 degree Celsius water bath for 16 to 20 hours. Be sure that the slides are in the dark while hybridizing.

7. Post hybridization Washes

7.1. Prepare the post hybridization buffers:

7.1.1. Wash 1:1×SSC+0.2% SDS

For 500 ml:

25 ml of 20×SSC

10 ml of 10% SDS, pH 7.2

465 ml of dH2O

7.1.1.1. Wash 2: 0.1×SSC+0.2% SDS

For 500 ml:

2.5 ml of 20×SSC

10 ml of 10% SDS, pH 7.2

487.5 ml of dH2O

7.1.1.2. Wash 3: 0.1% SSC

For 500 ml:

2.5 ml of 20×SSC

497.5 ml of dH2O

7.2. Prepare enough falcon tubes for a series of 5 washes of each slide (one wash with Wash 1, one wash with Wash 2 and three washes with Wash 3. Be sure that you use a new tube for each slide for each wash).

7.3. Place 40-50 ml of Wash 1 into falcon tubes and place into a 42 degree Celsius, shaking incubator to pre warm Wash 1. (This incubator should be in the dark or at least minimal light)

7.4. While Wash 1 is warming, prepare the rest of the falcon tubes with 40-50 ml of Wash solution for the rest of the washes.

Once slides are ready to be washed, the following steps must be done in the dark or very minimal light.

7.5. Transfer slides from incubation chamber to a falcon tube containing 50 ml of the pre-heated Wash 1. Place in a shaking 42 degree Celsius incubator for 4 minutes.

7.6. Transfer slides from Wash 1 to a falcon tube containing Wash 2. Place in a shaking incubator for 4 minutes. This and the rest of the following steps can be carried out at Room temp too.

7.7. Transfer slides from Wash 2 to the 1 st series of Wash 3. Place in a shaking incubator for 4 minutes.

7.8. Transfer slides from 1st series of Wash 3 to 2nd series of Wash 3. Place in a shaking incubator for 4 minutes.

7.9. Transfer slides from 2nd series of Wash 3 to 3rd series of Wash 3. Place in a shaking incubator for 4 minutes.

7.10. Transfer slides to slide rack and dry by centrifuging at 500 rpm for 5 minutes.

7.11. The slides are now ready to be scanned and must be scanned within an hour.

The slides are then processed with the SCANARRAY™ system, apparatus and protocol according to manufacturer's protocols to acquire the image of the microarray. The microarray images are further processed with the QUANTARRAY T system according to manufacturer's protocols.

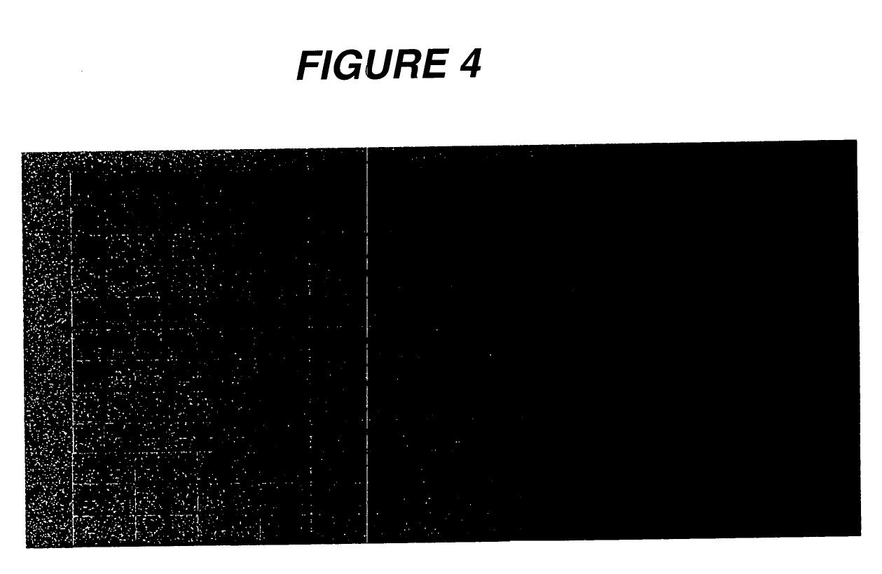

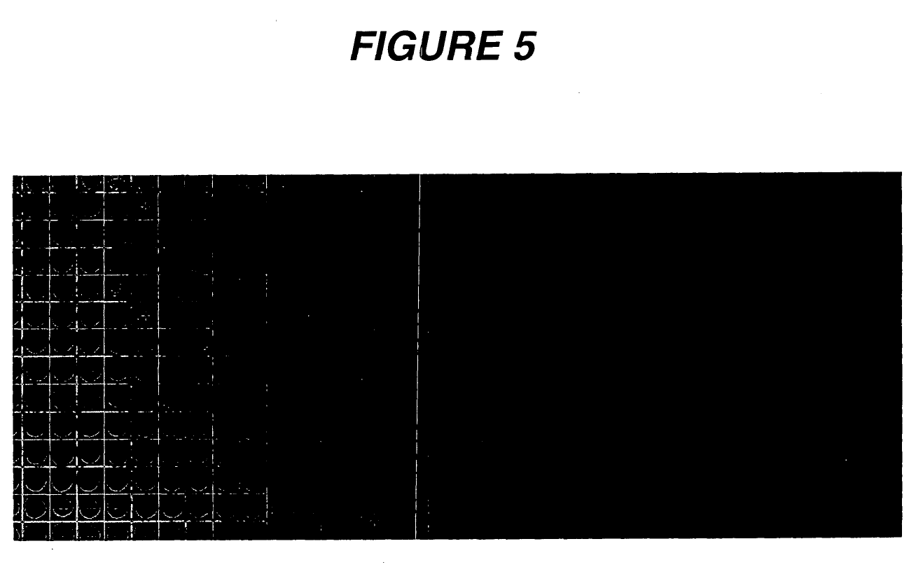

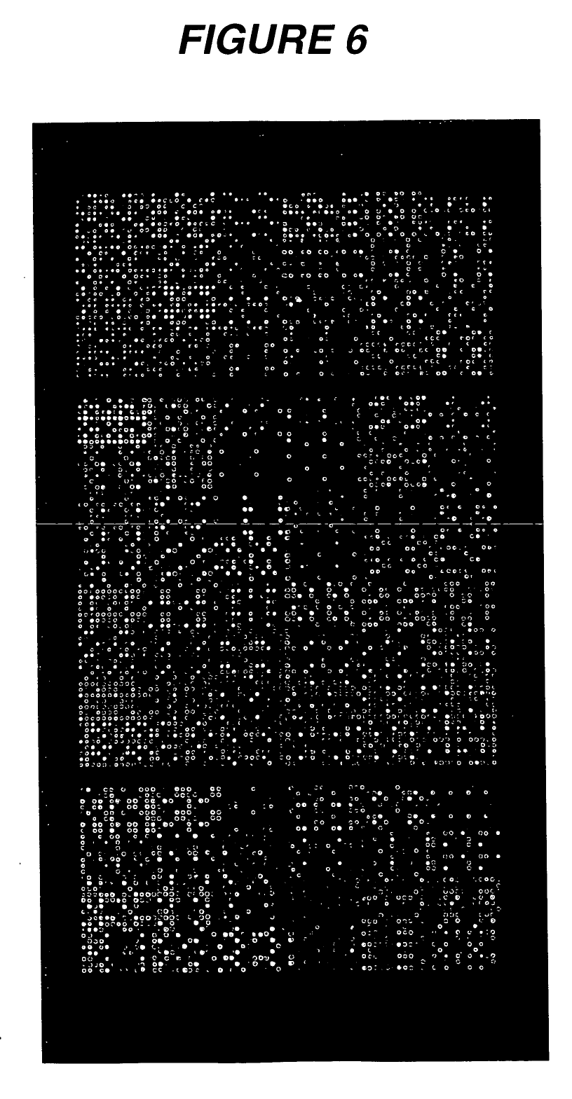

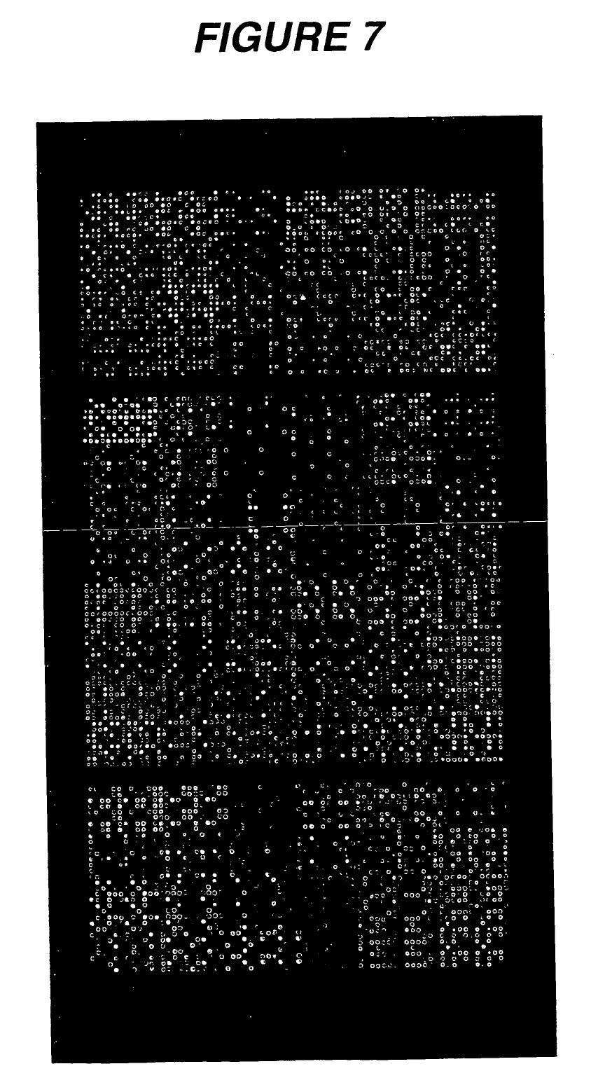

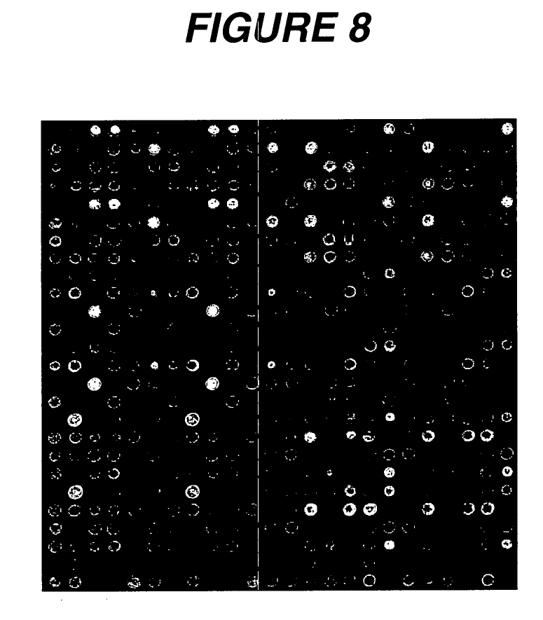

Examples of microarray images produced using the techniques and compositions described herein are set forth in FIGS. 4, 5, 6, 7, and 8.

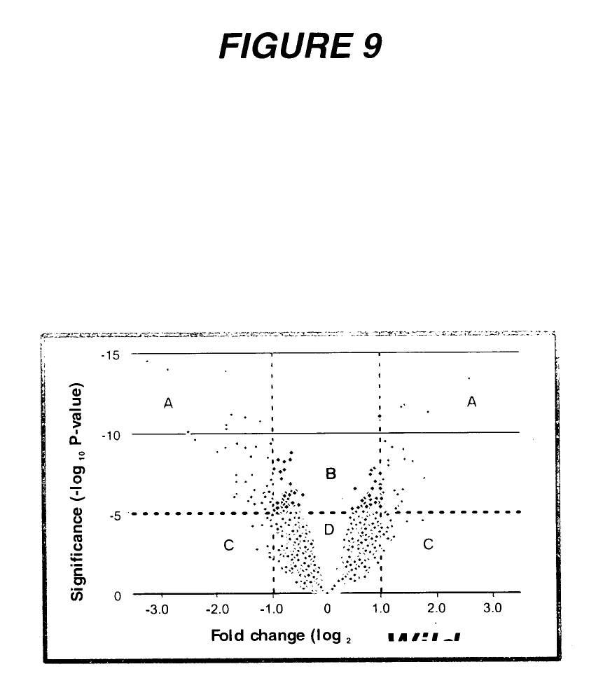

Based on a solid experimental design strategy (as proposed by Kerr and Churchill, 2001), addressing each scientist's specific biological hypotheses, and by using cutting edge statistical analytical tools (Wolfinger et al., 2001), it is possible to identify genes that have very small fold changes, but demonstrates highly significant biological relevance. This is only possible if the data that enters the analytical process is of superior quality, as demonstrated by using the present optimized microarray procedure. Based on the experimental design strategy, and the superior quality raw expression data generated using our approach, we are able to identify candidate genes that are highly significantly differentially regulated, but completely confounded to experimental error (false positives or negatives). Referring now to FIG. 9, these are the genes indicated in section C. In addition, we are also able to identify genes, as previously pointed out, that are represented by less than a two fold change, but have a significant biological influence in the overall genetic network or metabolism (Section B). These genes have always been completely disregarded and considered to have no biological relevance.

Gene significance is usually estimated using the “mixed model system” (Wolfinger et al., 2001). This model shows that changes in gene expression less than two fold can be statistically significant (Jin et al., 2001). Briefly, the log₂ transformed data (yᵢⱼₖ) are subjected to a normalization model: yᵢⱼₖ=μ+Aᵢ+Dⱼ+(A×D)ᵢⱼ+εᵢⱼₖ, where μ is the sample mean, Aᵢ is the effect of the array, Dⱼ is the effect of the dye, (A×D)ᵢⱼ is the effect of the array-dye interaction, and εᵢⱼ is the stochastic error. The residual values from this model are then fit into a gene-specific model in the form of rᵢⱼₖ=μ+Aᵢ+Tⱼ+Nₖ+εᵢⱼₖ, where Tⱼ corresponds to the j^{th} treatment, and Nₖ is the effect of the clone position on the array. Both models were implemented using PROC MIXED in SAS (SAS institute Inc. SAS/STAT Software version 8, SAS Institute, Cary, N.C., 1999).

It will be understood that various details of the invention may be changed without departing from the scope of the invention. Furthermore, the foregoing description is for the purpose of illustration only, and not for the purpose of limitation, as the invention is defined by the claims as set forth hereinafter.

That which is claimed is:

1. A method of producing a population of labeled target cDNA, comprising combining a cDNA template with a mixture comprising 48 μM dATP, 48 μM dCTP, 48 μM dGTP, 6 μM dTTP and 6 μM of fluorescently labeled nucleotide selected from the group consisting of dUTP-Cy3™ and dUTP-Cy5™ to provide a nucleotide labeling mixture;
adding a nucleic acid primer sufficient to prime the enzymatic generation of a population of target nucleic acids complementary to the cDNA template; and then
reacting the primer and the nucleotide labeling mixture in the presence of the Klenow fragment of DNA polymerase I to produce labeled target cDNA.

2. The method according to claim 1, wherein the primer is the HEXANUCLEOTIDE™ primer.

3. The method according to claim 1, wherein the cDNA template is a first strand complement of an mRNA population.

4. The method according to claim 1, wherein the primer and the nucleotide labeling mixture in the presence of the Klenow fragment of DNA polymerase I are reacted at 37° C.

5. A method of hybridizing a population of target nucleic acids to an array made up of a plurality of probe nucleic acid samples stably associated with the surface of a solid support, said method comprising:
generating said population of target nucleic acids by:
combining a cDNA template with a mixture comprising 48 mM dATP, 48 mM dCTP, 48 mM dGTP, 6 mM dTTP and 6 mM of fluorescently labeled nucleotide selected from the group consisting of dUTP-Cy3™ and dUTP-Cy5™ to provide a nucleotide labeling mixture;
adding a nucleic acid primer sufficient to prime the enzymatic generation of a population of target nucleic acids complementary to the cDNA template; and then
reacting the primer and the nucleotide labeling mixture in the presence of the Klenow fragment of DNA polymerase I to produce labeled target cDNA; and then
hybridizing said generated population of target nucleic acids to plurality of probe nucleic acid samples stably associated with the surface of a solid support.

6. The method according to, wherein the probe nucleic acid samples comprise cDNA.

7. The method of claim 1, wherein the probe nucleic acid samples comprise oligonucleotides.

8. The method of claim 5, wherein the solid support is a glass slide.

9. The method of claim 8, wherein the glass slide is coated with an aminosilane compound.

10. The method of claim 5, wherein the hybridization step is carried out in a hybridization buffer comprising polyA RNA, Calf Thymus DNA, 5×SSC, 5× Denhard's solution, 50% formamide, and 0.5% SDS, wherein the SDS has a pH of between about 7.18 and about 7.25.

11. The method according to claim 10, wherein the SDS has a pH of 7.2.

12. The method according to claim 5, further comprising the step of treating the plurality of probe nucleic acid samples stably associated with the surface of a solid support with a prehybridizing buffer prior to hybridization, the prehybridizing buffer comprising 5×SSC, 1% BSA Fraction V and 0.1% SDS, and wherein the SDS has a pH of between about 7.18 and about 7.25.

13. The method according to claim 10, wherein the SDS has a pH of 7.2.

14. The method according to claim 5, wherein the hybridization step is carried out at 42° C.

15. The method according to claim 5, wherein the hybridization step is followed by at least three post-hybridization washes with post-hybridization buffers, wherein the first post-hybridization buffer comprises 1×SSC and 0.2% SDS, the second post-hybridization buffer comprises 0.1×SSC and 0.2% SDS, and the third post-hybridization buffer comprises 0.1% SSC, and wherein the SDS has a pH of between about 7.18 and about 7.25.

16. The method according to claim 15, wherein the SDS has a pH of 7.2.

17. A kit for fluorescently labeling a nucleic acid, comprising:
a labeling mixture comprising dATP, dCTP, dGTP, dTTP and at least one of a fluorescently labeled nucleotide selected from the group consisting of dUTP-Cy3™ and dUTP-Cy5™, wherein the ratio of dATP to dCTP to dGTP to dTTP to dUTP-Cy3 or dUTP-Cy5 is 8:8:8:1:1.

18. The kit according to claim 17, wherein the labeling mix is a 5× mixture, and ratio of concentrations of dATP to dCTP to dGTP to dTTP to dUTP-Cy3 or dUTP-Cy5 is 240 μM: 240 μM: 240 μM:30 μM:30 μM.

19. The kit according to claim 17, further comprising the HEXANUCLEOTIDE™ primer.

20. The kit according to claim 17, further comprising the Klenow fragment of DNA polymerase I in an amount sufficient to incorporate nucleotides into a cDNA strand in a reverse transcriptase reaction.
